(19) United States
(12) Patent Application Publication
(10) Pub. No.: US 2023/0242956 A1
(43) Pub. Date: Aug. 3, 2023

(54) POLYPHOSPHATE KINASE MUTANT, ENGINEERED STRAIN AND APPLICATION THEREOF

(71) Applicant: ZHEJIANG UNIVERSITY OF TECHNOLOGY, Hangzhou (CN)

(72) Inventors: Yaping XUE, Hangzhou (CN); Yuzhen XUE, Hangzhou (CN); Shijia ZHANG, Hangzhou (CN); Qi SHEN, Hangzhou (CN); Yuguo ZHENG, Hangzhou (CN)

(21) Appl. No.: 18/159,371

(22) Filed: Jan. 25, 2023

(30) Foreign Application Priority Data
Jan. 28, 2022 (CN) 202210104075.9

(51) Int. Cl. C12P 19/02; C12N 9/12

(52) U.S. Cl. CPC C12P 19/02; C12N 9/1229; C12Y 207/04001

(57) ABSTRACT

The present invention discloses a polyphosphate kinase mutant, engineered strain and application thereof, wherein the polyphosphate kinase mutant is obtained by single- or multi-site mutations of the amino acid at position 79, 106, 108, 111 or 285 of the amino acid sequence shown in SEQ ID No. 2. The present invention provides a variety of polyphosphate kinase mutants derived from Cytophaga hutchinsonii, and the specific enzyme activity of these mutants is 2.7-17.9 times higher than that of the parent polyphosphate kinase, more than 70% of the amount of adenosine triphosphate (ATP) consumption in ATP-dependent biocatalytic synthesis reactions may be reduced by the ATP regeneration system constituted by the mutants, which has broad industrial application prospects.

### TECHNICAL FIELD

The present invention belonging to the technical field of bioengineering, in particular relates to a polyphosphate kinase (PPK) mutant, engineering strain and application thereof, and develops an efficient and inexpensive ATP regeneration system.

### BACKGROUND ART

Adenosine triphosphate (ATP) is a key molecule in organisms that regulates various biological processes such as energy metabolism, RNA and DNA synthesis, and signal transduction. Activities of a large number of potential biocatalysts, including ligases, kinases, and synthetases, also depend on ATP. Introducing an ATP regeneration system during these biotransformation processes can significantly reduce ATP consumption. Most ATP regeneration systems include a phosphate donor and a phosphotransferase that catalyzes the interaction between ADP and the phosphate donor. However, the biosynthesis of many valuable products such as oxytetracycline, hexane-1,6-diamine, and β-carboline amide requires a regeneration system that directly generates ATP from AMP. In order to regenerate ATP from AMP, a regeneration system for synthesizing ADP from AMP may be introduced into a regeneration system for generating ATP from ADP.

Notably, the use of multiple enzymes and phosphate donors will complicate the original biotransformation process. Given these limitations, class III polyphosphate kinase 2 (PPK2-III) have been intensively studied because they can convert AMP to ATP. The accessibility and stability of phosphate donors are critical for the application of an ATP regeneration system. For an ATP regeneration system comprising PPK2-III, inorganic polyphosphate (polyP) is a stable phosphate donor. In many studies, a long-chain polyP is used as phosphate donor. However, using a short-chain polyP as phosphate donor may make an ATP-generating system more attractive because short-chain polyP is cheaper than long-chain polyP. The most available polyP is polyphosphoric acid (PPA) which is a mixture of linear polyphosphoric acids with varying lengths, PPA is extensively used in the manufacture of pigments, petroleum catalysts, fragrances and flame retardants. Herein, we aim to develop an efficient and inexpensive ATP regeneration system which is based on PPK2-III and capable of taking AMP and PPA as substrates. Molecular modification is carried out on polyphosphate kinase derived from Cytophaga hutchinsonii (ChPPK) to improve its enzymatic properties for the construction of the inexpensive and efficient ATP regeneration system.

### SUMMARY OF THE INVENTION

Aiming at the problem that the activity of the existing polyphosphate kinase is not high, the present invention provides a polyphosphate kinase mutant and constructs an ATP regeneration system by taking a genetically recombinant strain or crude enzyme solution of the polyphosphate kinase mutant as biocatalyst to solve the problem of low efficiency of the existing ATP regeneration system.

Technical solutions adopted in the present invention are as follows:

The present invention provides a polyphosphate kinase mutant, wherein the polyphosphate kinase mutant is obtained by single- or multi-site mutations of the amino acid at position 79, 106, 108, 111 and 285 of the amino acid sequence shown in SEQ ID No. 2; the nucleotide sequence of the encoding gene of the amino acid sequence shown in SEQ ID No. 2 is shown in SEQ ID No. 1.

Preferably, the polyphosphate kinase mutant is obtained by subjecting the amino acid sequence shown in SEQ ID NO: 2 to one of the following mutations: (1) mutating alanine at position 79 into glycine (A79G); (2) mutating serine at position 106 into cysteine (S106C); (3) mutating isoleucine at position 108 into phenylalanine, asparagine or tyrosine (1108F, 1108N, I108Y); (4) mutating serine at position 111 into glutamic acid, lysine or alanine (S111E, S111K, S111 Å ); (5) mutating leucine at position 285 into proline (L285P); (6) mutating alanine at position 79 into glycine and isoleucine at position 108 into phenylalanine (A79G/I108F); (7) mutating alanine at position 79 into glycine, serine at position 106 into cysteine and isoleucine at position 108 into phenylalanine (A79G/S106C/1108F); (8) mutating alanine at position 79 into glycine, serine at position 106 into cysteine, isoleucine at position 108 into phenylalanine and serine at position 111 into alanine (A79G/S106C/I108F/S111 Å ); or (9) mutating alanine at position 79 into glycine, serine at position 106 into cysteine, isoleucine at position 108 into phenylalanine and leucine at position 285 into proline (A79G/S106C/1108F/L285P).

The present invention also provides an encoding gene of the polyphosphate kinase mutant, a recombinant vector comprising the encoding gene, and a recombinant genetically engineered strain prepared by transforming the recombinant vector into a host cell; the vector is a pET expression vector, a pCW expression vector or a pPIC expression vector, preferably a plasmid pET-28a(+); the host cell is Escherichia coli, Bacillus subtilis, Streptomyces, Saccharomyces cerevisiae, Pichia or mammalian cell, preferably Escherichia coli (E. coli) BL21 (DE3).

The present invention also provides an application of the polyphosphate kinase mutant in constructing an ATP regeneration system, the application is as follows: the polyphosphate kinase mutant and polyphosphoric acid (PPA) are used to substitute partial ATP, the polyphosphate kinase mutant acts in the form of a crude enzyme solution or pure enzyme extracted from wet cells, in which the wet cells are obtained by fermentation culture of the genetically engineered strain containing the polyphosphate kinase mutant. The polyphosphate kinase mutant of the present invention is capable of substituting partial ATP in all ATP-dependent biotransformation reactions.

The present invention also provides an application of the polyphosphate kinase mutant in synthesizing β-nicotinamide mononucleotide (NMN), the application is carried out as follows: supernatants as catalysts, adenosine triphosphate (ATP) and nicotinamide ribose (NR) as substrates, magnesium chloride, polyphosphoric acid (PPA) and a pH6.5 buffer as a reaction medium are used to carry out a reaction at 37° C. (preferably for 6h), thereby obtaining β-nicotinamide mononucleotide (NMN); in which, the supernatants are obtained by resuspension and subsequent ultrasonication of the wet cells in a buffer, wherein the wet cells are obtained by respective induction culture of the genetically engineered strain containing the polyphosphate kinase mutant and the genetically engineered strain containing the nicotinamide riboside kinase (NRK).

The amount of the adenosine triphosphate (ATP) calculated by the volume of the buffer is 10-100 mM, preferably is 25 mM; the amount of the nicotinamide ribose (NR) calculated by the volume of the buffer is 50-200 mM, preferably is 100 mM; the amount of the magnesium chloride calculated by the volume of the buffer is 5-20 mM, preferably is 10 mM; the amount of the polyphosphoric acid (PPA) calculated by the volume of the buffer is 1-10 g/L, preferably is 4.8 g/L; the amount of the supernatant containing the polyphosphate kinase mutant, calculated by the weight of the wet cells, per unit volume of the buffer is 2-30 mg (the weight of the wet cells)/mL(buffer), preferably is 4 mg (the weight of the wet cells)/mL (buffer); and the amount of the supernatant containing the nicotinamide riboside kinase, calculated by the weight of the wet cells, per unit volume of the buffer is 5−30 mg (the weight of the wet cells)/mL(buffer), preferably is 8 mg (the weight of the wet cells)/mL (buffer).

Preferably, the buffer is a pH 6.5, 50 mM potassium phosphate buffer.

Preferably, the catalyst is prepared by the following method: the genetically engineered strain containing the polyphosphate kinase mutant are inoculated into LB liquid medium containing 50 μg/mL kanamycin, cultured at 37° C. and 200 rpm for 12 h, the resulting inoculum is inoculated into fresh LB liquid medium containing 50 μg/mlkanamycin with 1% (v/v) incubating volume and cultured at 37° C. and 150 rpm, until OD600 of the cells reaches 0.6, isopropyl β-D-1-thiogalactopyranoside(IPTG) is added with a final concentration of 0.1 mM, and the bacteria solution is subjected to induction culture at 28° C. for 12 hours; the resulting solution is subjected to centrifugation at 4° C. and 8000 rpm for 10 min, the resulting supernatant is discarded and sediment is collected, thereby obtaining the wet cells; the collected wet cells are resuspended in a pH7.2, 50 mM potassium phosphate buffer(PBS) (preferably with 40 g wet cells/L buffer) and subjected to a ultrasonication machine for cell disruption at 50W for 20 min with a pattern of 1 s on, 2 s off, thereby obtaining a cell lysate solution; the cell lysate solution is subjected to centrifugation at 12000 g for lmin, and the supernatant is collected as a crude enzyme solution.

Preferably, the method for preparing the supernatant of the genetically engineered strain containing the nicotinamide riboside kinase (NRK) is the same as that for the supernatant of the genetically engineered strain containing the polyphosphate kinase mutant. The amino acid sequence of the NRK is shown in SEQ ID NO: 4, the nucleotide sequence is shown in SEQ ID NO: 3, the vector for constructing the engineered strain is pET-28a(+), and the host is E. coli BL21 (DE3).

The present invention also provides an application of the polyphosphate kinase mutant in synthesizing glucose-6-phosphate (G6P), and the application is carried out as follows: supernatants as catalysts, adenosine triphosphate (ATP) and glucose as substrates, magnesium chloride, polyphosphoric acid (PPA) and a pH7.2, 50 mM potassium phosphate buffer as a reaction medium are used to carry out a reaction at 37° C. (preferably for 8h), thereby obtaining G6P; in which, the supernatants are obtained by respective induction culture, resuspension of the resulting wet cells in a buffer and subsequent ultrasonication of the genetically engineered strain containing the polyphosphate kinase mutant and the genetically engineered strain containing the hexokinase (HK).

The amount of the adenosine triphosphate (ATP) calculated by the volume of the buffer is 10-100 mM, preferably is 25 mM; the amount of the glucose calculated by the volume of the buffer is 20-150 mM, preferably is 100 mM; the amount of the magnesium chloride calculated by the volume of the buffer is 5-20 mM, preferably is 10 mM; the amount of the polyphosphoric acid (PPA) calculated by the volume of the buffer is 1-10 g/L, preferably is 4.8 g/L; the amount of the supernatant containing the polyphosphate kinase mutant, calculated by the weight of the wet cells, per unit volume of the buffer is 2-30 mg (the weight of the wet cells)/mL(buffer), preferably is 4 mg (the weight of the wet cells)/mL(buffer); and the amount of the supernatant containing the hexokinase (HK), calculated by the weight of the wet cells, per unit volume of the buffer is 5−30 mg (the weight of the wet cells)/mL(buffer), preferably is 12 mg (the weight of the wet cells)/mL(buffer).

Preferably, the buffer is a pH 7.2, 50 mM potassium phosphate buffer.

Preferably, the method for preparing the supernatant of the genetically engineered bacteria containing the hexokinase (HK) is the same as that for the supernatant of the genetically engineered bacteria containing the polyphosphate kinase mutant. The amino acid sequence of the HK is shown in SEQ ID NO: 6, the nucleotide sequence is shown in SEQ ID NO: 5, the vector for constructing the engineered bacteria is pET-28a(+), and the host is E. coli BL21 (DE3).

Compared with prior art, advantages of the present invention are mainly embodied in: The present invention provides a variety of polyphosphate kinase mutants derived from Cytophaga hutchinsonii. The specific enzyme activity of these mutants is 2.7-17.9 times higher than that of the parent polyphosphate kinase. The ATP regeneration system constituted by these mutants can reduce more than 70% of the amount of ATP in the synthesis of NMN and G6P without affecting the final yield. Therefore, the present invention has broad industrial application prospects.

### BRIEF DESCRIPTION OF THE DRAWINGS

FIG. 1: the relative enzyme activity of the crude enzyme solution containing the mutant prepared in Example 4.

FIG. 2: the relative enzyme activity of the mutant prepared in Example 5.

FIG. 3: the relative enzyme activity of the mutant prepared in Example 6.

FIG. 4: the effect of temperature on the enzyme activity of the mutant ChPPK/A79G/S106C/I108F/L285P and the wild-type enzyme in Example 7.

FIG. 5: the effect of pH on the enzyme activity of the mutant ChPPK/A79G/S106C/I108F/L285P and the wild-type enzyme in Example 7.

FIG. 6: the effect of the concentration of AMP on the enzyme activity of the mutant ChPPK/A79G/S106C/I108F/L285P and the wild-type enzyme in Example 7.

FIG. 7: the effect of the concentration of PPA on the enzyme activity of the mutant ChPPK/A79G/S106C/I108F/L285P and the wild-type enzyme in Example 7.

FIG. 8: a curve diagram of the yields of NMN in different reaction systems in Example 9.

FIG. 9: a histogram of the yields of G6P in different reaction systems in Example 10.

### SPECIFIC EMBODIMENTS

The present invention is further illustrated below with specific examples, but the protection scope of the present invention is not limited thereto:

In the following examples, the experimental methods without specific experimental conditions are usually carried out in accordance with conventional conditions, such as the conditions described in Molecular Cloning: A Laboratory Manual (Third Edition, J. Sambrook et al.).

LB agar plate is composed of 10 g/L tryptone, 10 g/L sodium chloride, 5 g/L yeast extract, 15 g/L agar and water as a solvent, and the pH is natural.

LB liquid medium is composed of 10 g/L tryptone, 10 g/L sodium chloride, 5 g/L yeast extract and water as a solvent, and the pH is natural.

### Example 1: Construction of Wild-Type E. coli BL21 (DE3)-ChPPK

A PPK protein sequence derived from Cytophaga hutchinsonii (ChPPK, GenBank accession number: ABG57400.1) in GenBank was optimized for E. coli codon preference and fused to a C-terminal 6His tag. The resulting recombinant sequence of the gene ChPPK with a length of 930 bp was synthesized by Tsingke Biotechnology Co., Ltd. (Beijing, China), the nucleotide sequence is shown in SEQ ID NO: 1, and the amino acid sequence of the encoded protein is shown in SEQ ID NO: 2.

The recombinant gene ChPPK was inserted after T7 promoter of pET-28a(+) to obtain an expression plasmid pET28-ChPPK. The expression plasmid was transformed into E. coli BL21 (DE3), plated on LB plates containing 50 μg/mL kanamycin and cultured at 37° C. for 8-12h, and positive colonies were picked, which were wild-type E. coli BL21 (DE3)-ChPPK for expression of recombinant ChPPK.

SEQ ID NO: 1
ATGGCAACCGATTTTAGCAAACTGAGCAAATATGT
TGAAACGCTGCGTGTGAAACCGAAACAGAGCATTG
ATCTGAAAAAGGATTTTGATACCGATTATGATCAT
AAAATGCTGACGAAAGAAGAAGGTGAAGAACTGCT
GAATCTGGGTATTAGTAAACTGAGCGAAATTCAGG
AAAAACTGTATGCATCTGGCACAAAAAGCGTGCTG
ATTGTTTTTCAGGCAATGGATGCAGCAGGTAAAGA
TGGTACCGTTAAACATATTATGACGGGTCTGAATC
CGCAGGGTGTTAAAGTGACCAGCTTTAAAGTTCCG
TCCAAAATTGAACTGAGTCATGATTATCTGTGGCG
TCATTATGTGGCACTGCCGGCAACCGGCGAAATTG
GTATTTTTAACCGTAGCCATTATGAAAATGTGCTG
GTTACCCGTGTACATCCGGAATATCTGCTGAGCGA
ACAGACCAGCGGTGTTACCGCAATTGAACAGGTAA
ATCAGAAATTTTGGGATAAACGCTTTCAGCAGATC
AATAACTTTGAACAGCATATTAGCGAAAACGGTAC
CATTGTTCTGAAATTTTTTCTGCATGTTTCCAAAA
AGGAACAGAAAAAGCGTTTTATTGAACGTATCGAA
CTGGATACCAAAAATTGGAAATTTTCAACCGGTGA
TCTGAAAGAACGTGCCCATTGGAAAGATTATCGTA
ATGCGTATGAAGATATGCTGGCAAATACCTCTACC
AAACAGGCCCCGTGGTTTGTTATTCCGGCCGATGA
TAAATGGTTTACCCGTCTGCTGATTGCAGAAATTA
TCTGTACCGAACTGGAAAAACTGAATCTGACCTTT
CCGACCGTGAGCCTGGAACAGAAAGCGGAACTGGA
AAAAGCAAAAGCAGAACTGGTTGCAGAAAAATCAA
GCGATCATCATCATCACCACTAA.
SEQ ID NO: 2
MATDFSKLSKYVETLRVKPKQSIDLKKDFDTDYDH
KMLTKEEGEELLNLGISKLSEIQEKLYASGTKSVL
IVFQAMDAAGKDGTVKHIMTGLNPQGVKVTSFKVP
SKIELSHDYLWRHYVALPATGEIGIFNRSHYENVL
VTRVHPEYLLSEQTSGVTAIEQVNQKFWDKRFQQI
NNFEQHISENGTIVLKFFLHVSKKEQKKRFIERIE
LDTKNWKFSTGDLKERAHWKDYRNAYEDMLANTST
KQAPWFVIPADDKWFTRLLIAEIICTELEKLNLTF
PTVSLEQKAELEKAKAELVAEKSSDHHHHHH.

### Example 2: Induction Expression of the Wild-Type E. coli BL21 (DE3)-ChPPK and Extraction of the Wild-Type Polyphosphate Kinase

(1) Crude enzyme solution: the wild-type E. coli BL21 (DE3)-ChPPK obtained in Example 1 was inoculated into LB liquid medium containing 50 μg/mL kanamycin, and cultured at 37° C. and 200 rpm for 12 h, the resulting inoculum was inoculated into fresh LB liquid medium containing 50 μg/ml kanamycin with 1% (v/v) incubating volume and cultured at 37° C. and 150 rpm until OD600 of the cells reached 0.6, IPTG was added with the final concentration of 0.1 mM, and the bacteria solution was subjected to induction culture at 28° C. for 12 hours; the resulting solution was subjected to centrifugation at 4° C. and 8000 rpm for 10 min, the resulting supernatant was discarded and sediment was collected, thereby obtaining the wet cells containing recombinant ChPPK. The collected wet cells were resuspended in a pH7.2, 50 mM potassium phosphate buffer saline(PBS) with 40 g wet cell s/L buffer and subjected to a ultrasonication machine for cell disruption at 50W for 20 min with 1 s breaking each 2 s pause, thereby obtaining a cell lysate solution; the cell lysate solution was subjected to centrifugation at 12000 g for lmin, and the supernatant was collected as a crude enzyme solution. The amount of the crude enzyme solution used in the following examples was calculated by the amount of the cells in the potassium phosphate buffer before ultrasonication.

(2) Pure enzyme: 5 mL of the crude enzyme solution was diluted with 40 mL of potassium phosphate buffer (20 mM, pH 7.2), and then applied to a HisTrap HP purification column of GE Healthcare (10 mL column, pre-washed with a pH 7.2, 20 mM potassium phosphate buffer containing 500 mM NaCl). The resulting purification column was eluted with 100 mL of washing buffer (a pH7.2, 20 mM potassium phosphate buffer containing 500 mM sodium chloride and 50 mM imidazole) at a rate of 0.5 mL/min to remove the absorbed protein impurities, and then eluted with an elution buffer (a pH7.2, 20 mM potassium phosphate buffer containing 500 mM sodium chloride and 50 mM imidazole) at a speed of 0.5 mL/min to collect an elution solution containing the target protein. Then the elution solution containing the target protein was was dialyzed in a dialysis bag (the MWCO of the dialysis bag was 14 KDa) with a pH7.2 of 20 mM potassium phosphate buffer for 48h, and the retentate was taken as pure enzyme. The concentration of the pure enzyme was determined by Beyotime BCA Protein Assay Kit(P0012), and the amount of the pure enzyme used in the following examples was calculated by protein content.

### Example 3: Determination of Enzyme Activity

0.4 mg of the crude enzyme solution prepared by the method in Example 2 or 0.05 mg of the pure enzyme, polyphosphoric acid (PPA) at a final concentration of 1.6 g/L, adenosine phosphate (AMP) at a final concentration of 2.25 mM, and MgCl₂ at a final concentration of 10 mM were added to 10 mL of 50 mM potassium phosphate buffer (pH 7.5). The reaction solution was incubated at 37° C. for 5 min, and then 10 mL of 0.2 M phosphoric acid solution was added to terminate the reaction. The ATP content in the solution was determined by HPLC method.

The instrument used for HPLC was an Agilent 1260 Infinity II (Agilent Technologies Co., Ltd., USA), equipped with an Agilent 2414 UV detector, an Agilent 1525 pump, and an Agilent 717 injector. The column was an)(Bridge C18 column (C18, 5 μm, 4.6×250 mm, Waters, California, USA). The flow rate of the mobile phase was 1 mL/min, the UV detection wavelength was 254 nm, the mobile phase was potassium phosphate buffer (50 mM, pH 7.0), the injection volume was 10 μL, and the run time was 9 min. The peak area data obtained by injection of different concentrations of ATP (0.125 mM, 0.25 mM, 0.5 mM, 1.0 mM, 2.0 mM and 4.0 mM) were used to obtain a standard curve of ATP concentration and peak area. The curve equation is y=(x+189.78)/3847 (R²=0.998), wherein y is the ATP concentration (mM), and x is the ATP peak area obtained from the liquid phase.

Definition of enzyme activity: An enzyme activity is defined as the amount of enzyme required to generate 1 μmol ATP per minute (the first 5 minutes) under the above conditions.

The enzyme activities of the crude enzyme solution and the pure enzyme solution in Example 2 were 0.0029 U and 0.0932 U, respectively.

### Example 4: Identification of Key Sites in ChPPK Through Alanine Scanning Mutagenesis

1. Screening of Mutation Sites

The substrate PPA was represented by polyP containing 5 phosphate monomers. Docking of ChPPK and the substrate (AMP and the polyP containing 5 phosphate monomers) was performed by Autodock vina 1.1.2. According to the binding results of the enzyme and the substrate, most of the amino acids(D77, G80, K81, D82, F102, K103, V104, P105, R117, R133, E137, N138, V141 and R208) within 5 Å from the substrate and some amino acids(5106, 1108 and S111) within 12 Å from the substrate were selected. The plasmid pET28-ChPPK in Example 1 was used as a template, and the primers listed in Table 1 were used to mutate each amino acid of the above sites into alanine by the Quick-change mutagenesis method. The PCR system for mutagenesis contained 25 μl of 2×Phanta Max mixture, 0.4 μM of each of forward primer and reverse primer, about 10 ng of the template, and purified water in a total volume of 50 PCR conditions were as follows: an initial denaturation step at 95° C. for 1 min, followed by 20 amplification cycles, each of which included 95° C. for 10 s, 55° C. for 30 s and 72° C. for 5 min, and finally 72° C. for 5 min.

TABLE 1
Mutation sites and primers

| Site | Primer | Primer sequence |
|---|---|---|
| 77 | Forward | TCAGGCAATGGCTGCAGCAGGTAAAGAT |
| | Reverse | ACCTGCTGCAGCCATTGCCTGAAAAACA |
| 80 | Forward | ATGCAGCAGCTAAAGATGGTACCGTT |
| | Reverse | ACCATCTTTAGCTGCTGCATCCATTG |
| 81 | Forward | AGCAGGTGCAGATGGTACCGTTAAACATAT |
| | Reverse | TACCATCTGCACCTGCTGCATCCATTG |
| 82 | Forward | AGGTAAAGCTGGTACCGTTAAACAT |
| | Reverse | ACGGTACCAGCTTTACCTGCTGCA |
| 102 | Forward | TGACCAGCGCTAAAGTTCCGTCCAAAAT |
| | Reverse | ACGGAACTTTAGCGCTGGTCACTTTAACA |
| 103 | Forward | ACCAGCTTTGCAGTTCCGTCCAAAATT |
| | Reverse | ACGGAACTGCAAAGCTGGTCACTTT |
| 104 | Forward | AGCTTTAAAGCTCCGTCCAAAATTGAAC |
| | Reverse | TTGGACGGAGCTTTAAAGCTGGTCA |
| 105 | Forward | TTTAAAGTTGCGTCCAAAATTGAACTGAG |
| | Reverse | ATTTTGGACGCAACTTTAAAGCTGG |
| 106 | Forward | AAGTTCCGGCCAAAATTGAACTGAGT |
| | Reverse | TCAATTTTGGCCGGAACTTTAAAGCTGG |
| 108 | Forward | TCCGTCCAAAGCTGAACTGAGTCATGATTA |
| | Reverse | ACTCAGTTCAGCTTTGGACGGAACTT |
| 111 | Forward | TTGAACTGGCTCATGATTATCTGTGGC |
| | Reverse | TAATCATGAGCCAGTTCAATTTTGGACG |
| 117 | Forward | TATCTGTGGGCTCATTATGTGGCAC |
| | Reverse | ACATAATGAGCCCACAGATAATCATGA |
| 133 | Forward | TTTTTAACGCTAGCCATTATGAAAATGTGC |
| | Reverse | AATGGCTAGCGTTAAAAATACCAATTTCG |
| 137 | Forward | AGCCATTATGCAAATGTGCTGGTTAC |
| | Reverse | AGCACATTTGCATAATGGCTACGGTTAAAAA |
| 138 | Forward | ATTATGAAGCTGTGCTGGTTACCCGT |
| | Reverse | ACCAGCACAGCTTCATAATGGCTAC |
| 141 | Forward | TGTGCTGGCTACCCGTGTACATCC |
| | Reverse | TACACGGGTAGCCAGCACATTTTCATA |
| 208 | Forward | TTATTGAAGCTATCGAACTGGATACCAA |
| | Reverse | AGTTCGATAGCTTCAATAAAACGCTT |

2. Mutant Engineering Strain and Crude Enzyme Solution

The mutated plasmid obtained in step 1 was transformed into a host strain E. coli BL21 (DE3), the crude enzyme solution was prepared by the method of Example 2, and the relative enzyme activity was determined by the method of Example 3. As the results shown in FIG. 1, taking the activity of the wild-type crude enzyme solution as 100%, the supernatant of the lysate containing the ChPPK mutant of which residue D77, D82, R133, E137 or R208 was mutated into alanine completely lost its activity, indicating that these sites are conserved amino acids which are unsuitable for evolutionary research. The enzyme activity changed to some extent when the residues G80, K81, F102, K103, P105, S106, 1108, S111 and R117 were mutated to alanine, which proves that these sites have a great influence on the activity of ChPPK, so saturation mutagenesis were carried out at these sites in Example 5.

### Example 5: Enhance the Activity of ChPPK by Saturation Mutagenesis

1. Site-Directed Saturation Mutagenesis

In addition to the candidate sites in Example 4, A79 and L285 were also selected for saturation mutagenesis for the following reasons: in the binding model of ChPPK to substrate, the A79 site is within 5 Å from the polyP. Since the site of the original sequence is alanine, site A79 is unnecessary to carry out alanine mutation in Example 4. In Example 5, saturation mutagenesis on A79 was directly performed. In addition, L285 is a key site that determines the lid domain of ChPPK, so saturation mutagenesis was also performed in Example 5.

The plasmid pET28-ChPPK in Example 1 was used as a template, and the primers listed in Table 2 were used to perform saturation mutagenesis on selected sites A79, G80, K81, F102, K103, P105, S106, 1108, S111, and R117 by the Quick-change mutagenesis method.

TABLE 2
Mutation sites and primers

| Site | Primer | Primer sequence |
|---|---|---|
| 79 | Forward | TGGATGCANNKGGTAAAGATGGTAC |
| | Reverse | TCTTTACCMNNTGCATCCATTGCC |
| 80 | Forward | ATGCAGCANNKAAAGATGGTACCGTT |
| | Reverse | ACCATCTTTMNNTGCTGCATCCATTG |
| 81 | Forward | AGCAGGTNNKGATGGTACCGTTAAACATAT |
| | Reverse | TACCATCMNNACCTGCTGCATCCATTG |
| 102 | Forward | TGACCAGCNNKAAAGTTCCGTCCAAAAT |
| | Reverse | ACGGAACTTTMNNGCTGGTCACTTTAACA |
| 103 | Forward | ACCAGCTTTNNKGTTCCGTCCAAAATT |
| | Reverse | ACGGAACMNNAAAGCTGGTCACTTT |
| 105 | Forward | TTTAAAGTTNNKTCCAAAATTGAACTGAG |
| | Reverse | ATTTTGGAMNNAACTTTAAAGCTGG |
| 106 | Forward | AGTTCCGNNKAAAATTGAACTGAGT |
| | Reverse | TCAATTTTMNNCGGAACTTTAAAGCTGG |
| 108 | Forward | CGTCCAAANNKGAACTGAGTCATGA |
| | Reverse | TCAGTTCMNNTTTGGACGGAACTTTAAAG |
| 111 | Forward | TTGAACTGNNKCATGATTATCTGTGGC |
| | Reverse | TAATCATGMNNCAGTTCAATTTTGGACG |
| 117 | Forward | TATCTGTGGNNKCATTATGTGGCAC |
| | Reverse | ACATAATGMNNCCACAGATAATCATGA |
| 285 | Forward | ACCGTGAGCNNKGAACAGAAAGCGG |
| | Reverse | TTCTGTTCMNNGCTCACGGTCGGAAA |

In Table 2, N = A, T, G, C; K = G, T; M = A, C.

2. Mutant Engineering Strain and Crude Enzyme Solution

The mutated plasmid obtained in step 1 was transformed into a host strain E. coli BL21 (DE3), the pure enzyme was prepared by the method of Example 2, and the enzyme activity was measured by the method of Example 3.

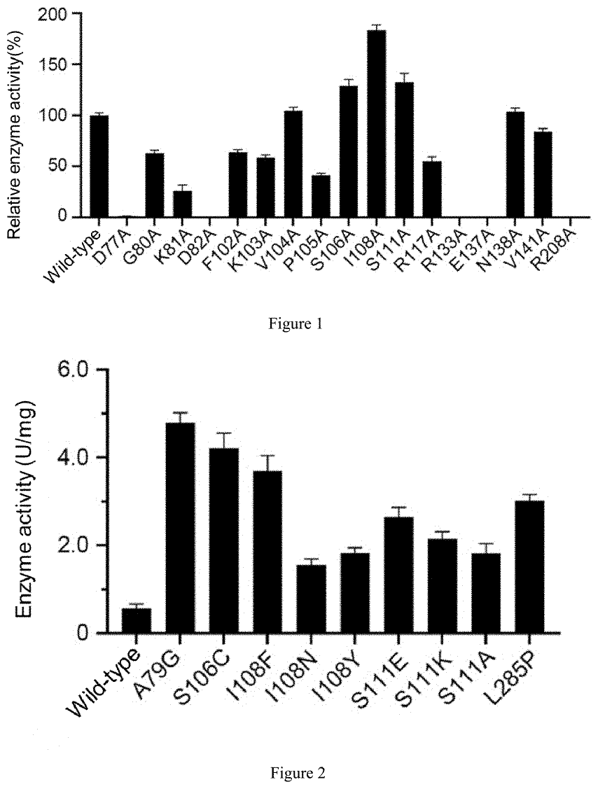

As the results shown in FIG. 2, the enzyme activity of the mutant with single residue mutation A79G, S106C, 1108F, 1108N, 1108Y, S111E, S111K, S111 Å or L285P was significantly improved.

### Example 6: Enhance the Activity of ChPPK Via Combinations of Beneficial Mutations

1. Double Mutation, Triple Mutation

The sites where single residue mutation can increase the enzyme activity are located at positions 79, 106, 108, 111 and 285 of the amino acid sequence of ChPPK. According to the docking results of the enzyme and the substrate, positions 79, 106, 108 and 111 are located in the substrate binding pocket, while the position 285 is far away from these substrate binding sites. Therefore, it is less likely that position 285 interacts with other beneficial mutations. Hence, firstly, combined mutations were carried out at positions 79, 106, 108 and 111. In order to combine the beneficial mutations at positions 79, 106, 108 and 111, PCR was performed to obtain a fragment containing all possibly beneficial mutations at positions 79, 106, 108 and 111. The PCR template was the plasmid pET28-ChPPK, primers were PPK-M-b primer F, PPK-M-b primer R1 and PPK-M-b primer R2. Among them, PPK-M-b primer F was the forward primer, and a mixture of PPK-M-b primer R1 and PPK-M-b primer R2 (in a molar ratio of 1:1 added in the PCR system) was the reserve primer. Degenerate bases were included in the forward primers and the reserve primers to generate all possibly beneficial mutations.

PPK-M-b primer F:
TCAGGCAATGGATGCAGSAGGTAAAGATGGTA;
PPK-M-b primer R1:
ACAGATAATCATGCTYCAGTTCAWWTTTASACGGAACₒ
PPK-M-b primer R2:
ACAGATAATCATGTGMCAGTTCAWWTTTASACGGAAC
Degenerate bases
(S = G, C; Y = C, T; W = A, T; M = A, C)

To religate this fragment, which contains all possibly beneficial mutations at positions 79, 106, 108, and 111, into the plasmid, a plasmid pET28-ChPPK without this fragment was amplified by PCR. The primers were as follows:

pET-PPK primer F: CATGATTATCTGTGGCGTCATTATGTG;
pET-PPK primer R: TGCATCCATTGCCTGAAAAACAATCAGₒ

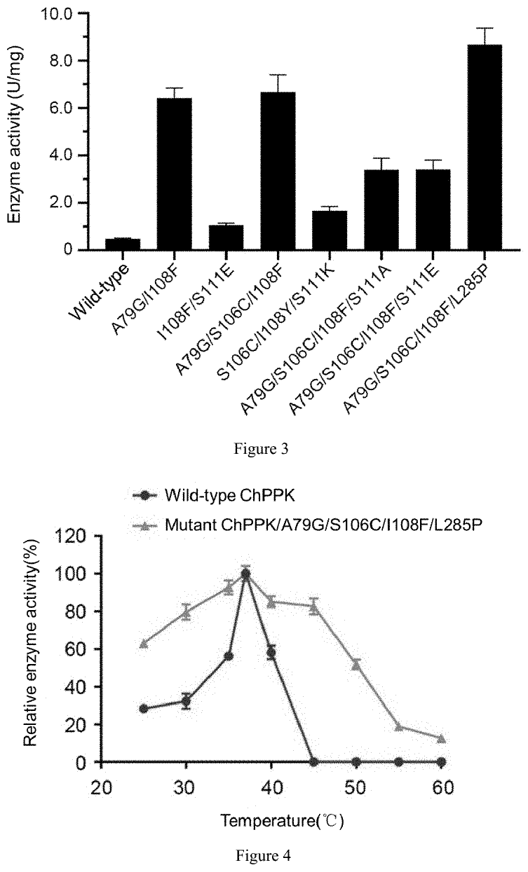

After ligating the plasmid fragment with the fragments containing all possibly beneficial mutations at positions 79, 106, 108 and 111, the resulting plasmid was transformed into a host strain E. coli BL21 (DE3), and the pure enzyme was prepared by the method of Example 2, and the enzyme activity was determined by the method of Example 3. As the results shown in FIG. 3, the ChPPK with two mutations A79G/S108F and the ChPPK with three mutations A79G/S106C/I108F have higher enzyme activities. The ChPPK with two mutations I108F/S111E and the ChPPK with three mutations S106C/I108Y/S111K have lower enzyme activities.

2. Four Mutations

The beneficial mutation S111 Å mutant was further introduced into the plasmid pET28-ChPPK with three mutations A79G/S106C/I108F. The plasmid pET28-ChPPK containing three mutations A79G/S106C/I108F as the template, and the amplification primer for mutating alanine at position 111 in Table 1 was used for mutagenesis by Quick-change mutagenesis method. After transforming the mutated plasmid into a host strain E. coli BL21 (DE3), screening, expression and purification were carried out as described in Examples 1, 2 and 3. As the results shown in FIG. 3, the enzyme activity of the ChPPK with four mutations A79G/S106C/I108F/S111 Å was significantly lower than that of the ChPPK with three mutations A79G/S106C/I108F. In order to investigate the combined effect of the other beneficial mutations at position S111 and the pET28-ChPPK with three mutations A79G/S106C/I108F, the plasmid pET28-ChPPK with A79G/S106C/I108F as the template, position 111 was mutated to glutamic with the following primers by the Quick-change mutation method.

Primers used to mutate serine at position 111 to glutamic acid:

Forward primer: TTGAACTGGAACATGATTATCTGTGGC;
Reverse primer: TAATCATGTTCCAGTTCAATTTTGGACGₒ

The enzyme activity of the ChPPK with four mutations A79G/S106C/I108F/S111K was significantly lower than that of the ChPPK with three mutations A79G/S106C/I108F/, indicating that combination of all beneficial mutations at positions 79, 106, 108 and 111 does not produce the best results.

The L285P mutation was further introduced into the plasmid pET28-ChPPK with three mutations A79G/S106C/I108F. The plasmid pET28-ChPPK with three mutations A79G/S106C/I108F as a template, the following primers were used for mutagenesis by Quick-change mutagenesis method. After transforming the above mutated plasmid into a host strain E. coli BL21(DE3), screening, expression and purification were carried out as described in Examples 1, 2 and 3. As the results shown in FIG. 3, the mutant with four mutations A79G/S106C/I108F/L285P (ChPPK/A79G/S106C/I108F/L285P) showed the highest enzyme activity.

Mutation at Position 285

Forward primer: ACCGTGAGCCCAGAACAGAAAGCGG;
Reverse primer: TTCTGTTCTGGGCTCACGGTCGGAAAₒ

### Example 7: Characterization of the Mutant ChPPK/A79G/S106C/I108F/L285P

We compared the influences of temperature, pH and substrate concentration on the enzyme activities of wild-type ChPPK and mutant ChPPK/A79G/S106C/I108F/L285P.

1.Temperature

The engineering strain E. coli BL21(DE3)-ChPPK and E. coli BL21(DE3)-ChPPK/A79G/S106C/I108F/L285P constructed by the methods of Examples 1 and 6 were used to prepare pure enzymes by the method of Example 2. The enzyme activity was measured by the method of Example 3, and the temperature for measuring the enzyme activity was respectively changed to 25° C., 30° C., 35° C., 37° C., 40° C., 45° C., 50° C., 55° C. or 60° C.

As the results shown in FIG. 4, although 37° C. is the optimal reaction temperature for the wild-type ChPPK and the mutant ChPPK/A79G/S106C/I108F/L285P, the mutant ChPPK/A79G/S106C/I108F/L285P exhibited significantly higher relative activity at 42-50° C. For example, the mutant ChPPK/A79G/S106C/I108F/L285P retained 82% of enzyme activity at 45° C., whereas the wild-type ChPPK lost 96% of enzyme activity under this condition.

2, pH

The engineered strain E. coli BL21(DE3)-ChPPK and E. coli BL21(DE3)-ChPPK/A79G/S106C/I108F/L285P constructed by the methods of Examples 1 and 6 were used to prepare pure enzymes by the method of Example 2. The enzyme activity was measured by the method of Example 3, and the pH of the buffer was changed to 5.0-6.0 (50 mM citric acid-sodium citrate buffer), 6.0-8.0 (50 mM potassium phosphate buffer), 8.0-9.0 (50 mM borax-boric acid buffer) or 9.0-10.0 (50 mM glycine-NaOH buffer).

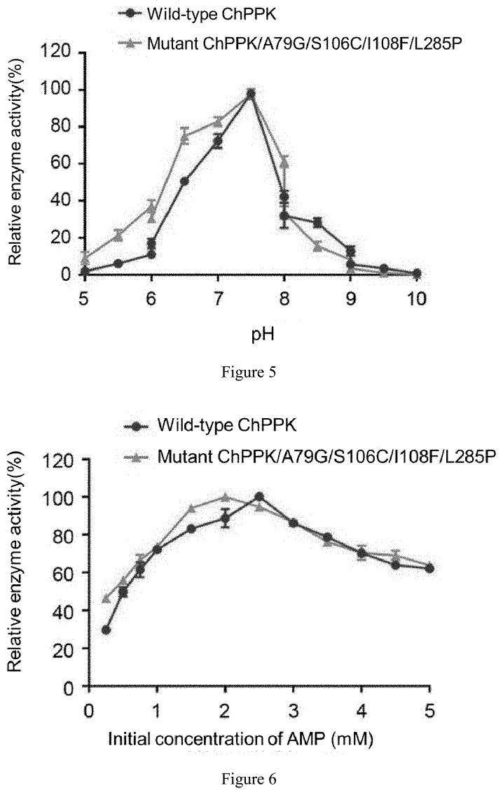

As the results shown in FIG. 5, the relative enzyme activities of the wild-type ChPPK and the mutant ChPPK/A79G/S106C/I108F/L285P reached a maximum at pH 7.5. However, the mutant ChPPK/A79G/S106C/I108F/L285P exhibited higher relative enzyme activity under acidic conditions.

3. AMP Concentration

The engineered strain E. coli BL21(DE3)-ChPPK and E. coli BL21(DE3)-ChPPK/A79G/S106C/I108F/L285P constructed by the methods of Examples 1 and 6 were used to prepare pure enzymes by the method of Example 2. The enzyme activity was measured by the method of Example 3, and the AMP concentration was respectively changed to 0.25 mM, 0.50 mM, 0.75 mM, 1.00 mM, 1.50 mM, 2.00 mM, 2.50 mM, 3.00 mM, 3.50 mM, 4.00 mM, 4.50 mM or 5.00 mM.

As the results shown in FIG. 6, the wild-type ChPPK and the mutant ChPPK/A79G/S106C/I108F/L285P reached the highest relative enzyme activities at AMP concentrations of 2.0-2.5 mM.

4. PPA Concentration

The engineered strain E. coli BL21(DE3)-ChPPK and E. coli BL21(DE3)-ChPPK/A79G/S106C/I108F/L285P constructed by the methods of Examples 1 and 6 were used to prepare pure enzymes by the method of Example 2. The enzyme activity was measured by the method of Example 3, and the PPA concentration was respectively changed to 0.32 g/L, 0.64 g/L, 0.96 g/L, 1.28 g/L, 1.60 g/L, 1.92 g/L, 2.24 g/L, 2.56 g/L, 2.88 g/L or 3.20 g/L.

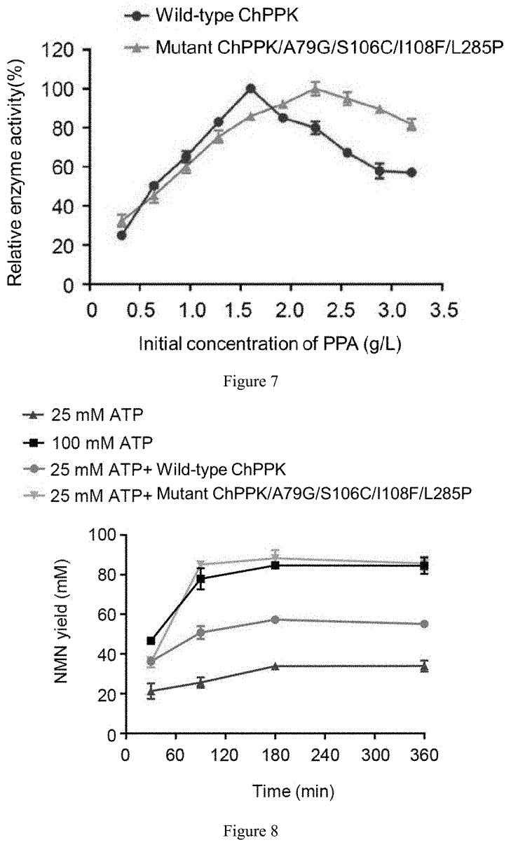

As the results shown in FIG. 7, the optimal PPA concentration for the wild-type ChPPK was 1.6 g/L, while the mutant ChPPK/A79G/S106C/I108F/L285P exhibited the highest relative enzyme activity in the presence of 2.24 g/L PPA, indicating that the mutant ChPPK/A79G/S106C/I108F/L285P has higher PPA tolerance.

### Example 8: Determination of Kinetic Parameters of the Wild-Type ChPPK and the Mutant ChPPK/A79G/S106C/I108F/L285P with Pure Enzymes

The wild-type Ecoli.BL21(DE3)-ChPPK constructed by the method of Example 1 and the engineered strain Ecoli.BL21(DE3)-ChPPK/A79G/S106C/I108F/L285P constructed by the method of Example 6 were used to prepare pure enzymes by the method of Example 2. Km and Kcat were calculated by pseudo-one-substrate kinetic model. In order to calculate the kinetics of the enzyme for the two substrates separately, the method of Example 3 was used to measure the enzyme activity, the reactions were under the condition of a fixed PPA concentration (1.6 g/L) with an adjustable AMP concentration (0, 0.25, 0.5, 0.75, 1.0, 1.5, 2.0, 2.5, 3.0, 3.5, 4.0, 4.5, 5.0 mM) or a fixed AMP concentration (5 mM) with an adjustable PPA concentration (0.32, 0.64, 0.96, 1.28, 1.6, 1.92, 2.24, 2.56, 2.88, 3.2 g/L).

The Km values of the wild-type ChPPK for AMP and PPA were similar to those of the mutant ChPPK/A79G/S106C/I108F/L285P (Shown in Table 3 and Table 4). The increased enzyme activity of the mutant ChPPK/A79G/S106C/I108F/L285P can be explained by its increased turnover number (kcat value) for AMP and PPA. The catalytic efficiency (kcat/Km) of the mutant ChPPK/A79G/S106C/I108F/L285P for AMP and PPA was 16-fold and 18-fold higher than that of the wild-type ChPPK, respectively.

TABLE 3
Kinetic parameters of wild-type ChPPK and mutant
ChPPK/A79G/S106C/I108F/L285P for substrate AMP

| Enzyme | Km (mM) | kcat (S⁻¹) | kcat/Km (s⁻¹ · mM⁻¹) |
|---|---|---|---|
| Wild-type | 1.66 ± 0.10 | 1.01 ± 0.05 | 0.61 |
| Mutant | 1.67 ± 0.18 | 16.33 ± 1.78 | 9.78 |

TABLE 4
Kinetic parameters of wild-type ChPPK and mutant
ChPPK/A79G/S106C/I108F/L285P for substrate PPA

| Enzyme | Km (g/L) | kcat (S⁻¹) | kcat/Km (s⁻¹ · (g/L)⁻¹) |
|---|---|---|---|
| Wild-type | 22.82 ± 0.42 | 10.44 ± 3.04 | 0.45 |

### Example 9: Application of Mutant ChPPK/A79G/S106C/1108F/L285P in NMN Biosynthesis

1. Crude Enzyme Solution of Nicotinamide Riboside Kinase

Since nicotinamide riboside kinase can catalyze the biosynthesis of nicotinamide ribose (NR) to nicotinamide mononucleotide (NMN), the amino acid sequence of nicotinamide riboside kinase (NRK, GenBank No.: XP 035204248.1) derived from Oxyura jamaicensis was used as a template to artificially synthesize a nicotinamide riboside kinase gene which was optimized for E. coli codons. The nucleotide sequence is shown in SEQ ID NO: 3, and the amino acid sequence is shown in SEQ ID NO: 4.

SEQ ID NO: 3
ATGAAATACATCATCGGTATCGGTGGTGTTACCAA
CGGTGGCAAAACCACCCTGACAAATCGTCTGGTTA
AAGCACTGCCTAACTGTTGTGTGGTTCACCAGGAC
GATTTTTTTAAACCTCAGGATCAGATTGAAGTTGG
TGAAGATGGCTTTAAACAATGGGACGTTCTGGACT
CTCTGGATATGGAAGCAATGGTTAGCACCGTTCGT
GCATGGATTGAAAATCCGGTTAAATTTGCACGTAG
CCACGGTGTTAATGTTACACCGGGCAGCAAAGAAC
CGGCAAGCAAAGATATTCATATTCTGGTTATTGAG
GGATTTCTGCTGTATAATTATAAACCGCTGATTGA
CCTGTTTGATATTCGTTATTATCTGGCAGTCCCTT
ATGATGAATGTAAACGTCGTCGTAGCACCCGTAAC
TATACCGTTCCGGATCCGCCGGGTCTGTTCGATGG
CCATGTTTGGCCGATGTATCTGAAACATCGTAAAG
AAATGGAAGACAATGGGGTGGATGTGGTTTATCTG
GATGGCCTGAAAAGCCGCGATGAACTGTACAACCA
GGTCTTTGAAGATATTCAGAATAAACTGCTGAACT
GCCTGCATCATCATCACCACCATTAA.ₒ
SEQ ID NO: 4
MKYIIGIGGVTNGGKTTLTNRLVKALPNCCVVHQD
DFFKPQDQIEVGEDGFKQWDVLDSLDMEAMVSTVR
AWIENPVKFARSHGVNVTPGSKEPASKDIHILVIE
GFLLYNYKPLIDLFDIRYYLAVPYDECKRRRSTRN
YTVPDPPGLFDGHVWPMYLKHRKEMEDNGVDVVYL
DGLKSRDELYNQVFEDIQNKLLNCLHHHHHHₒ

The NRK gene (shown in SEQ ID NO: 3) optimized for E. coli codons was inserted after the T7 promoter of pET-28a(+) to obtain an expression plasmid pET28-NRK. The expression plasmid was transformed into E. coli. BL21 (DE3), plated on LB plates containing 50 μg/mL kanamycin at 37° C. for 8-12 h, and positive clones were picked, which is wild-type E. coli BL21 (DE3)-NRK for expression of recombinant NRK. The crude enzyme solution was prepared by the method of Example 2, and the amount of the NRK was calculated by the amount of the cells before ultrasonication.

2. Biosynthesis of NMN

Reaction 1 (25 mM ATP): ATP at a final concentration of 25 mM, NR at a final concentration of 100 mM, MgCl₂ at a final concentration of 10 mM, and 8 mg/mL crude enzyme solution of the NRK were added into 1 mL of potassium phosphate buffer (50 mM, pH 6.5) to react at 37° C. for 6 h, and samples were taken at 0.5 h, 1.5 h, 3 h and 6 h for HPLC detection as the method of Example 3 (the retention time of NMN was 2.9 minutes). Peak area were obtained by injecting different concentrations of NMN (0.0625 mM, 0.125 mM, 0.25 mM, 0.5 mM, 1.0 mM and 2.0 mM) to calculate the standard curve of NMN, the curve equation is y=(x+254.3)/4587 (R²=0.995), wherein y is the concentration of NMN (mM), and x is the peak area of NMN. As the time-NMN yield profile of Reaction 1 shown in FIG. 8, the NMN yield was 34 mM after 6 hours.

Reaction 2 (100 mM ATP): The final concentration of ATP in Reaction 1 was changed to 100 mM, and the other operations were the same. As the results shown in FIG. 8, when the concentration of ATP reached 100 mM, the NMN yield increased to 85 mM after 6 h.

Reaction 3 (25 mM ATP+wild-type ChPPK): 4 mg/mL crude enzyme solution of the wild-type ChPPK prepared by the method of Example 2 and 4.8 g/L of PPA were added to Reaction 1, and the other operations were the same. The NMN concentration changes is shown in the FIG. 8, when the ATP regeneration system containing the wild-type ChPPK was introduced into the biosynthesis of NMN, the NMN yield increased to 55 mM after 6h.

Reaction 4 (25 mM ATP+mutant ChPPK/A79G/S106C/1108F/L285P): 4 mg/mL crude enzyme solution of the mutant ChPPK/A79G/S106C/1108F/L285P prepared by the method of Example 6 and 4.8 g/L of PPA were added to Reaction 1, the other operations were the same. As the results shown in FIG. 8, when the ATP regeneration system containing the mutant ChPPK/A79G/S106C/1108F/L285P was introduced into the biosynthesis of NMN, the NMN yield increased to 86 mM after 6h, which is indicating that the ATP regeneration system containing the mutant could save 75% of the ATP input without affecting the final NMN yield.

### Example 10: Application of Mutant ChPPK/A79G/S106C/1108F/L285P in G6P Biosynthesis

1. Crude Enzyme Solution of Hexokinase

Since hexokinase can catalyze the biosynthesis of glucose 6 phosphate (G6P) from glucose, the amino acid sequence of hexokinase (HK, GenBank No.: NP_013551.1) derived from Saccharomyces cerevisiae was used as a template to artificially synthesize the hexokinase gene which was optimized for E. coli codons. The nucleotide sequence is shown in SEQ ID NO: 5, and the amino acid sequence is shown in SEQ ID NO: 6.

SEQ ID NO: 5
ATGACCATTGAAAGCACCCTGGCACGCGAACTGGA
AAGTCTGATTCTGCCGGCGGATAGCATTGTGAATG
TGGTGGATCAGTTTCAGGAAGAACTGCTGAGCCGC
CTGCAGACCAACACCATTAGCATGCTGCCGCAGTG
CCTGGTGCCGGATAAACGCAGCCGCTGGAATCCGG
AAGATAAAATTCTGACCATTGATTTTGGTGGTACC
CGTCTGAAATTTGCGATTATTAGCCTTCCGCAGAT
TGTGATTGAATACAACGATGCGTTTGAACTGACCT
ATAACATTGTGGATTCAAATTTCTTTAACCAGATC
ATTTATACCATTTGCACCCGCCTGGCCGCCAATGG
TTATATCAAAAAAAAAAACGAAAGCTCAGAAGCGT
CAAAATTTTTTGTGAGCGTGACCTTTAGCTTTCCG
CTGAACCCGGAAGGCGAAGTGGTGGCGATGGGCAA
AGGTTTTGTGATGACCGATACCCTGCAGGGCAGCA
CCGTGAAACAGCTGATTCAGAGCAGCTTTCATCGC
ATTATTAGCGAGAATATTGAAGAGTTTTTTTGCAC
CATGAATGTGTGTCATGTGATTAATGATGCCATTG
CCGTGAGCCTGACCAGCAAATTTATTTGTGAAAAC
GATAGCATCAGCCTGATTATTGGCACCGGTACCAA
TGCGTGCTTTGAAGTGCCGTATGGCTATCTGCCGC
CGTTTAAACGCGATGCGCTGCGCGAAACCCTGCCG
AGCAGCTACAACAAAGAAACCCTGAATTTTAAACA
TGTGCTGATCAACAGCGAAATCGGCTTTATTGGCA
AAAATGTCATTGCGCTGCAGCCGTTTGATATTCAC
GGCGCAATTAGCTATGAAATGCCGCTGGAATGCGT
GACCAGCGGCAAATGGCTGCCGCTGAGCCTGAAAA
ACATTCTGCTGCAATATAATATTATTCCGAAAAAT
TTTCCGGTTGAATTTAATGGAGAACTGGTGTGCCA
GCTGGCGGAAGATTGCACCAATGCGTGGTTTGAAA
ATGAACATTATGCCCTGATTTGCCAGATTGCGCGC
CTGTTGATTAAACGCGCAGCGTTCTACGTGGCGGC
CATTGTGCAGGCGATTGATATTATCACCGGCTGCA
AAAATTATAATTTTATTCACATTGGCTATGTGGGC
TCATTTCTGCATAACAGCAACTTTTACCGTGAACA
GATTAAATATTATAGCAGCATTCACATTAAACTGC
AGTTCCTGAATCACTCAAATCTGCTGGGTGCGGCC
ATTGCCACCTACCTGAATAAATCAGATAACCAGGT
GCAGTAA
SEQ ID NO: 6
MTIESTLARELESLILPADSIVNVVDQFQEELLSR
LQTNTISMLPQCLVPDKRSRWNPEDKILTIDFGGT
RLKFAIISLPQIVIEYNDAFELTYNIVDSNFFNQI
IYTICTRLAANGYIKKKNESSEASKFFVSVTFSFP
LNPEGEVVAMGKGFVMTDTLQGSTVKQLIQSSFHR
IISENIEEFFCTMNVCHVINDAIAVSLTSKFICEN
DSISLIIGTGTNACFEVPYGYLPPFKRDALRETLP
SSYNKETLNFKHVLINSEIGFIGKNVIALQPFDIH
GAISYEMPLECVTSGKWLPLSLKNILLQYNIIPKN
FPVEFNGELVCQLAEDCTNAWFENEHYALICQIAR
LLIKRAAFYVAAIVQAIDIITGCKNYNFIHIGYVG
SFLHNSNFYREQIKYYSSIHIKLQFLNHSNLLGAA
IATYLNKSDNQVQ.

The HK gene (nucleotide sequence shown in SEQ ID NO: 5) optimized for E. coli codons was inserted after the T7 promoter of pET-28a(+) to obtain the expression plasmid pET28-HK. The expression plasmid was transformed into Ecoli.BL21 (DE3), plated on LB plate containing 50 μg/mL kanamycin at 37° C. for 8-12h, and positive clones were picked, which is wild-type E. coli BL21 (DE3)-HK for expression of recombinant HK. The crude enzyme solution was prepared as the method of Example 2, and the amount of the crude enzyme solution of the HK was calculated by the amount of the cells before ultrasonication.

2. Biosynthesis of G6P

Reaction 1 (25 mM ATP): ATP at a final concentration of 25 mM, glucose at a final concentration of 100 mM, MgCl₂ at final concentration of 10 mM, and 12 mg/mL crude enzyme solution of HK were added to 5 mL of potassium phosphate buffer (50 mM, pH 7.5) at 30° C. for 8 h. 1 mL of reaction buffer was sampled after the enzyme was inactivated by heating at 70° C. for 15 minutes. After centrifuging at 12,000×g for 10 minutes, the supernatant was collected for HPLC analysis.

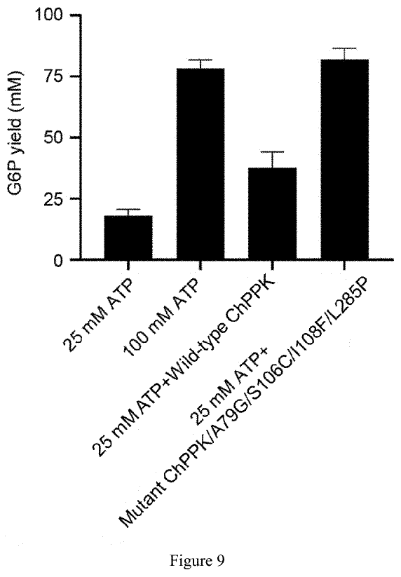

The instrument for HPLC detection of G6P was an Agilent 1260 Infinity II (Agilent Technologies Co., Ltd., USA), equipped with a Dionex ED40 detector, Agilent 1525 pump, Agilent 717 injector, the column was Dionex IonPac AS11-HC, and the detection temperature is 30° C. The mobile phase was an aqueous solution of sodium hydroxide at a flow rate of 1 mL/min. Gradient elution was carried out as follows: between 0 and 10 minutes, the eluant was an aqueous solution of sodium hydroxide with an increasing concentration from 0 mM to 25 mM; between 10 and 12 minutes, the eluant was an aqueous solution of sodium hydroxide with an constant concentration of 25 mM; between 12 and 15 minutes, the eluant was an aqueous solution of sodium hydroxide with an increasing concentration from 25 mM to 100 mM; between 15 and 17 minutes, the eluant was an aqueous solution of sodium hydroxide with an constant concentration of 100 mM; between 17 and 21 minutes, the eluant was an aqueous solution of sodium hydroxide with an decreasing concentration from 100 mM to 25 mM. Peak area were obtained by injecting different concentrations of G6P (0.125 mM, 0.25 mM, 0.5 mM, 1.0 mM, 2.0 mM and 4.0 mM) to calculate the standard curve of G6P, the curve equation is y=(x+544.7)/3856 (R²=0.997), wherein y is the concentration (mM) of G6P, and x is the peak area of G6P. The standard curve was used to calculate the G6P yield catalyzed by the HK crude enzyme solution. As shown in FIG. 9, the yield of G6P was 18 mM after 8h.

Reaction 2 (100 mM ATP): The final concentration of ATP in Reaction 1 was changed to 100 mM, and the other operations were the same. As the results shown in FIG. 9, when the ATP concentration reached 100 mM, the G6P yield increased to 78 mM after 8h.

Reaction 3 (25 mM ATP+wild-type ChPPK): 4 mg/mL crude enzyme solution of wild-type ChPPK prepared by the method of Example 2 and 4.8 g/L of PPA were added to Reaction 1, and the other operations were the same. As shown in FIG. 9, when the ATP regeneration system containing the wild-type ChPPK was introduced into the biosynthesis of G6P, the G6P yield was increased to 38 mM after 8h.

Reaction 4 (25 mM ATP+mutant ChPPK/A79G/S106C/1108F/L285P): 4 mg/mL crude enzyme solution of mutant ChPPK/A79G/S106C/1108F/L285P prepared by the method of Example 6 and 4.8 g/L of PPA were added to Reaction 1, the other operations were the same. As the results shown in FIG. 9, when the ATP regeneration system containing the mutant ChPPK/A79G/S106C/1108F/L285P was introduced into the biosynthesis of G6P, the production of G6P increased to 82 mM after 8h, which is indicating that the ATP regeneration system containing the mutants could save 75% of the ATP input without affecting the final G6P yield.

1. A polyphosphate kinase mutant, wherein the polyphosphate kinase mutant is obtained by single- or multi-site mutations of the amino acid at position 79, 106, 108, 111 and 285 of the amino acid sequence shown in SEQ ID No. 2.

2. A polyphosphate kinase mutant as claimed in claim 1, wherein the polyphosphate kinase mutant is obtained by subjecting the amino acid sequence shown in SEQ ID NO: 2 to one of the following mutations: (1) mutating alanine at position 79 into glycine; (2) mutating serine at position 106 into cysteine; (3) mutating isoleucine at position 108 into phenylalanine, asparagine or tyrosine; (4) mutating serine at position 111 into glutamic acid, lysine or alanine; (5) mutating leucine at position 285 into proline; (6) mutating alanine at position 79 into glycine and isoleucine at position 108 into phenylalanine; (7) mutating alanine at position 79 into glycine, serine at position 106 into cysteine and isoleucine at position 108 into phenylalanine; (8) mutating alanine at position 79 into glycine, serine at position 106 into cysteine, isoleucine at position 108 into phenylalanine and serine at position 111 into alanine; or (9) mutating alanine at position 79 into glycine, serine at position 106 into cysteine, isoleucine at position 108 into phenylalanine and leucine at position 285 into proline.

3. An encoding gene of the polyphosphate kinase mutant as claimed in claim 1.

4. A recombinant genetically engineered strain comprising the encoding gene as claimed in claim 3.

5. An application of the polyphosphate kinase mutant as claimed in claim 1 in constructing an ATP regeneration system.

6. An application of the polyphosphate kinase mutant as claimed in claim 1 in synthesizing f3-nicotinamide mononucleotide, wherein the application is carried out as follows: supernatants as catalysts, adenosine triphosphate and nicotinamide ribose as substrates, magnesium chloride, polyphosphoric acid and a pH6.5 buffer as a reaction medium are used to carry out a reaction at 37° C., thereby obtaining β-nicotinamide mononucleotide; in which, the supernatants are obtained by resuspension and subsequent ultrasonication of the wet cells in a buffer, wherein the wet cells are obtained by respective induction culture of the genetically engineered strain containing the polyphosphate kinase mutant and the genetically engineered strain containing the nicotinamide riboside kinase.

7. An application as claimed in claim 6, wherein the amount of the adenosine triphosphate calculated by the volume of the buffer is 10-100 mM; the amount of the nicotinamide ribose calculated by the volume of the buffer is 50-200 mM; the amount of the magnesium chloride calculated by the volume of the buffer is 5-20 mM; the amount of the polyphosphoric acid calculated by the volume of the buffer is 1-10 g/L; the amount of the supernatant containing the polyphosphate kinase mutant, calculated by the weight of the wet cells, per unit volume of the buffer is 2-30 mg/mL; and the amount of the supernatant containing the nicotinamide riboside kinase, calculated by the weight of the wet cells, per unit volume of the buffer is 5−30 mg/mL.

8. An application as claimed in claim 6, wherein the catalyst is prepared by the following method: the genetically engineered strain containing the polyphosphate kinase mutant are inoculated into LB liquid medium containing 50 μg/mL kanamycin, cultured at 37° C. and 200 rpm for 12 h, the resulting inoculum is inoculated into fresh LB liquid medium containing 50 μg/ml kanamycin with 1% (v/v) incubating volume and cultured at 37° C. and 150 rpm, until OD600 of the cells reaches 0.6, isopropyl β-D-1-thiogalactopyranoside is added with a final concentration of 0.1 mM, and the bacteria solution is subjected to induction culture at 28° C. for 12 hours; the resulting solution is subjected to centrifugation at 4° C. and 8000 rpm for 10 min, the resulting supernatant is discarded and sediment is collected, thereby obtaining the wet cells; the collected wet cells are resuspended in a pH7.2, 50 mM potassium phosphate buffer and subjected to a ultrasonication machine for cell disruption at 50W for 20 min with a pattern of 1 s on, 2 s off, thereby obtaining a cell lysate solution; the cell lysate solution is subjected to centrifugation at 12000 g for 1 min, and the supernatant is collected as a crude enzyme solution; the preparation method of the supernatant of the genetically engineered strain containing the nicotinamide riboside kinase is same as that of the genetically engineered strain containing the polyphosphate kinase mutant.

9. An application of the polyphosphate kinase mutant as claimed in claim 1 in synthesizing glucose-6-phosphate, wherein the application is carried out as follows: supernatants as catalysts, adenosine triphosphate and glucose as substrates, magnesium chloride, polyphosphoric acid and a pH7.2, 50 mM potassium phosphate buffer as a reaction medium are used to carry out a reaction at 37° C., thereby obtaining glucose-6-phosphate; in which, the supernatants are obtained by respective induction culture, resuspension of the resulting wet cells in a buffer and subsequent ultrasonication of the genetically engineered strain containing the polyphosphate kinase mutant and the genetically engineered strain containing the hexokinase.

10. An application as claimed in claim 9, wherein the amount of the adenosine triphosphate calculated by the volume of the buffer is 10-100 mM; the amount of the glucose calculated by the volume of the buffer is 20-150 mM; the amount of the magnesium chloride calculated by the volume of the buffer is 5-20 mM; the amount of the polyphosphoric acid calculated by the volume of the buffer is 1-10 g/L; the amount of the supernatant containing the polyphosphate kinase mutant, calculated by the weight of the wet cells, per unit volume of the buffer is 2-30 mg/mL; and the amount of the supernatant containing the hexokinase, calculated by the weight of the wet cells, per unit volume of the buffer is 5−30 mg/mL.


SEQUENCE LISTING (6 sequences)

SEQ ID NO: 1 - DNA, 933 bp, Cytophaga hutchinsonii
  source 1..933: mol_type = genomic DNA
atggcaaccgattttagcaaactgagcaaatatgttgaaacgctgcgtgtgaaaccgaaacagagcattgatctgaaaaaggattttgataccgattatgatcataaaatgctgacgaaagaagaaggtgaagaactgctgaatctgggtattagtaaactgagcgaaattcaggaaaaactgtatgcatctggcacaaaaagcgtgctgattgtttttcaggcaatggatgcagcaggtaaagatggtaccgttaaacatattatgacgggtctgaatccgcagggtgttaaagtgaccagctttaaagttccgtccaaaattgaactgagtcatgattatctgtggcgtcattatgtggcactgccggcaaccggcgaaattggtatttttaaccgtagccattatgaaaatgtgctggttacccgtgtacatccggaatatctgctgagcgaacagaccagcggtgttaccgcaattgaacaggtaaatcagaaattttgggataaacgctttcagcagatcaataactttgaacagcatattagcgaaaacggtaccattgttctgaaattttttctgcatgtttccaaaaaggaacagaaaaagcgttttattgaacgtatcgaactggataccaaaaattggaaattttcaaccggtgatctgaaagaacgtgcccattggaaagattatcgtaatgcgtatgaagatatgctggcaaatacctctaccaaacaggccccgtggtttgttattccggccgatgataaatggtttacccgtctgctgattgcagaaattatctgtaccgaactggaaaaactgaatctgacctttccgaccgtgagcctggaacagaaagcggaactggaaaaagcaaaagcagaactggttgcagaaaaatcaagcgatcatcatcatcaccactaa

SEQ ID NO: 2 - protein, 311 aa, Cytophaga hutchinsonii
MATDFSKLSKYVETLRVKPKQSIDLKKDFDTDYDHKMLTKEEGEELLNLGISKLSEIQEKLYASGTKSVLIVFQAMDAAGKDGTVKHIMTGLNPQGVKVTSFKVPSKIELSHDYLWRHYVALPATGEIGIFNRSHYENVLVTRVHPEYLLSEQTSGVTAIEQVNQKFWDKRFQQINNFEQHISENGTIVLKFFLHVSKKEQKKRFIERIELDTKNWKFSTGDLKERAHWKDYRNAYEDMLANTSTKQAPWFVIPADDKWFTRLLIAEIICTELEKLNLTFPTVSLEQKAELEKAKAELVAEKSSDHHHHHH

SEQ ID NO: 3 - DNA, 621 bp, Oxyura jamaicensis
  source 1..621: mol_type = genomic DNA
atgaaatacatcatcggtatcggtggtgttaccaacggtggcaaaaccaccctgacaaatcgtctggttaaagcactgcctaactgttgtgtggttcaccaggacgatttttttaaacctcaggatcagattgaagttggtgaagatggctttaaacaatgggacgttctggactctctggatatggaagcaatggttagcaccgttcgtgcatggattgaaaatccggttaaatttgcacgtagccacggtgttaatgttacaccgggcagcaaagaaccggcaagcaaagatattcatattctggttattgagggatttctgctgtataattataaaccgctgattgacctgtttgatattcgttattatctggcagtcccttatgatgaatgtaaacgtcgtcgtagcacccgtaactataccgttccggatccgccgggtctgttcgatggccatgtttggccgatgtatctgaaacatcgtaaagaaatggaagacaatggggtggatgtggtttatctggatggcctgaaaagccgcgatgaactgtacaaccaggtctttgaagatattcagaataaactgctgaactgcctgcatcatcatcaccaccattaa

SEQ ID NO: 4 - protein, 206 aa, Oxyura jamaicensis
MKYIIGIGGVTNGGKTTLTNRLVKALPNCCVVHQDDFFKPQDQIEVGEDGFKQWDVLDSLDMEAMVSTVRAWIENPVKFARSHGVNVTPGSKEPASKDIHILVIEGFLLYNYKPLIDLFDIRYYLAVPYDECKRRRSTRNYTVPDPPGLFDGHVWPMYLKHRKEMEDNGVDVVYLDGLKSRDELYNQVFEDIQNKLLNCLHHHHHH

SEQ ID NO: 5 - DNA, 1302 bp, Saccharomyces cerevisiae
  source 1..1302: mol_type = genomic DNA
atgaccattgaaagcaccctggcacgcgaactggaaagtctgattctgccggcggatagcattgtgaatgtggtggatcagtttcaggaagaactgctgagccgcctgcagaccaacaccattagcatgctgccgcagtgcctggtgccggataaacgcagccgctggaatccggaagataaaattctgaccattgattttggtggtacccgtctgaaatttgcgattattagccttccgcagattgtgattgaatacaacgatgcgtttgaactgacctataacattgtggattcaaatttctttaaccagatcatttataccatttgcacccgcctggccgccaatggttatatcaaaaaaaaaaacgaaagctcagaagcgtcaaaattttttgtgagcgtgacctttagctttccgctgaacccggaaggcgaagtggtggcgatgggcaaaggttttgtgatgaccgataccctgcagggcagcaccgtgaaacagctgattcagagcagctttcatcgcattattagcgagaatattgaagagtttttttgcaccatgaatgtgtgtcatgtgattaatgatgccattgccgtgagcctgaccagcaaatttatttgtgaaaacgatagcatcagcctgattattggcaccggtaccaatgcgtgctttgaagtgccgtatggctatctgccgccgtttaaacgcgatgcgctgcgcgaaaccctgccgagcagctacaacaaagaaaccctgaattttaaacatgtgctgatcaacagcgaaatcggctttattggcaaaaatgtcattgcgctgcagccgtttgatattcacggcgcaattagctatgaaatgccgctggaatgcgtgaccagcggcaaatggctgccgctgagcctgaaaaacattctgctgcaatataatattattccgaaaaattttccggttgaatttaatggagaactggtgtgccagctggcggaagattgcaccaatgcgtggtttgaaaatgaacattatgccctgatttgccagattgcgcgcctgttgattaaacgcgcagcgttctacgtggcggccattgtgcaggcgattgatattatcaccggctgcaaaaattataattttattcacattggctatgtgggctcatttctgcataacagcaacttttaccgtgaacagattaaatattatagcagcattcacattaaactgcagttcctgaatcactcaaatctgctgggtgcggccattgccacctacctgaataaatcagataaccaggtgcagtaa

SEQ ID NO: 6 - protein, 433 aa, Saccharomyces cerevisiae
MTIESTLARELESLILPADSIVNVVDQFQEELLSRLQTNTISMLPQCLVPDKRSRWNPEDKILTIDFGGTRLKFAIISLPQIVIEYNDAFELTYNIVDSNFFNQIIYTICTRLAANGYIKKKNESSEASKFFVSVTFSFPLNPEGEVVAMGKGFVMTDTLQGSTVKQLIQSSFHRIISENIEEFFCTMNVCHVINDAIAVSLTSKFICENDSISLIIGTGTNACFEVPYGYLPPFKRDALRETLPSSYNKETLNFKHVLINSEIGFIGKNVIALQPFDIHGAISYEMPLECVTSGKWLPLSLKNILLQYNIIPKNFPVEFNGELVCQLAEDCTNAWFENEHYALICQIARLLIKRAAFYVAAIVQAIDIITGCKNYNFIHIGYVGSFLHNSNFYREQIKYYSSIHIKLQFLNHSNLLGAAIATYLNKSDNQVQ